(19) United States
(12) Patent Application Publication
(10) Pub. No.: US 2010/0152150 A1
(43) Pub. Date: Jun. 17, 2010

(54) THE APPLICATION OF MARINE STEROID IN PREPARING THE MEDICINE OF TREATING NEURONS DAMAGING

(76) Inventors: Guangmei Yan, Guangzhou (CN); Pengxin Qiu, Guangzhou (CN); Yijun Huang, Guangzhou (CN); Wei Yin, Guangzhou (CN); Xingwen Su, Guangzhou (CN); Ailing Liu, Guangzhou (CN); Hanfel Sang, Guangzhou (CN); Xiang Cai, Guangzhou (CN)

Correspondence Address: BRINKS HOFER GILSON & LIONE, P.O. BOX 10395, CHICAGO, IL, 60610, US

(21) Appl. No.: 12/376,217

(22) PCT Filed: Aug. 31, 2006
(86) PCT No.: PCT/CN06/02235

§ 371 Date: Jan. 4, 2010

(30) Foreign Application Priority Data
Aug. 3, 2006 (CN) 200610036926.1

(51) Int. Cl. A61K 31/575; C07J 9/00; A61P 25/16; A61P 25/28; A61P 9/00

(52) U.S. Cl. 514/182; 552/542

(57) ABSTRACT

The application of marine steroid, i.e. 24-methylene-cholest-3β,5α,6β,19-tetrol, in preparing the medicine of treating neurons damaging is provided in the present invention. The keto-sterols compounds marine steroid YC-1 extracted from Nephthea albida has the action of neuronal protection, and no toxic reaction under the effective protective dosage.

### FIELD OF THE INVENTION

This invention relates to the application of neuron related steroids, concretely the use of marine steroid YC-1 (24-methylene-cholest-3β,5α,6β,19-tetrol) in preparing medicines for treating neuron damages.

### BACKGROUND OF THE INVENTION

Cerebrovascular disease happens frequently and has become one of the three major diseases endangering human's health. Among annual death number caused by diseases, that caused by cerebrovascular disease ranks 2nd in Japan, 3rd in the US and has always been No. 1 in China in recent years. Cerebrovascular disease can be basically considered as a senile disease. Its incidence increases with age. For people above 55, the incidence doubles in every decade. As increasing of living standard, prolonging of longevity and intensifying of population aging, the incidence of cerebrovascular disease is constantly increasing. At the same time, sufferers of cerebrovascular disease are becoming younger and younger due to change of life style. Among cerebrovascular diseases, about 60-80% is caused by ischemia (abbr. cerebral ischemia). The main reason for cerebral ischemia is cerebral arteriosclerosis. The mechanism of cerebral arteriosclerosis is as follows: systemic arteriosclerosis usually happens first, followed by general atherosclerosis, angiostenosis and small vessel occlusion, which reduces the blood supply for brain parenchyma, even causes cerebral thrombosis, complete vessel occlusion and extensive acute infarct of relevant blood supplying brain tissues, which is called stroke or apoplexy, this will finally lead to brain dysfunction or even permanent function losing. The high incidence, morbidity, mortality, recurrence rate or cerebral ischemia and symptoms caused by them, like hemiplegia, aphasia, dementia, have become heavy burdens of modern families.

So far there are two kinds of major treatment for damages caused by cerebral ischemia: to improve and recover blood supply of the damaged brain tissue as soon as possible and/or protect the tissue from further injury of toxic metabolites. The aim of recanalization of the occlusive vessels as soon as possible is to supple blood for involved brain tissue timely, block the chain reaction of chemicals killing neurons, enhance the tolerance of brain tissue, and salvage functions of those tissue in penumbral area before irreversible damage happens. The protection of neurons can be achieved by blocking various harmful pathological process caused by ischemia, preventing brain damages induced by localized ischemia, reducing brain cell death and promoting functional recovery. If both of them are applied, they may cause synergistic effect, produce more pertinent and scientific therapy and improve the curative effect.

Means to improve and recover blood supply for ischemic brain tissue include thrombolysis, anticoagulation, and anti-platelet aggregation, et al.

### 1. Thrombolysis

Thrombolysis, especially those of ultra-early and high-dose, has become the first choice for ischemic brain damage since 1980s. Comparing with the placebo group, thrombolysis significantly increased the incidence of intracranial hemorrhage and other death. Therefore, the risk of thrombolysis is high and doctors have to master the indications and administration time, especially for areas of long-time ischemia, such as ischemic core and those vulnerable, for easy occurrence of reperfusion injury, postinfarction hemorrhage and severe brain edema.

### 2. Anticoagulation

Although anticoagulation can prevent the conversions of prothrombin to thrombin and fibrinogen to fibrin together with platelet aggregation, it has no direct effect on existing thrombosis. Thus, it is very important to apply anticoagulation as soon as possible. However some scholars hold different ideas. They think immediate anticoagulation (like heparin, low mocular heparin, heparin factors, or special thrombin inhibitors) at the moment of thrombosis didn't show long- or short-term improving effect. Although immediate therapy may reduce thrombosis in deep venous and pulmonary artery, this effect was counteracted by the risk of intra- and extra-cranial hemorrhage.

### 3. Anti-Platelet Aggregation

With function of anti-platelet aggregation, aspirin has been widely used on ischemic brain damage, but the dosage has not been unified during these years. Laboratory research proved that about 30-50 mg/d of aspirin is enough to be effective. Therefore, clinically low dose aspirin has been used for treatment and prevention of ischemic cerebrovascular disease. These years new anti-platelet medicine-ticlopidine-has been used on ischemic cerebrovascular disease and its clinical effect is recognized better than aspirin.

### 4. Hemodilution

Hemodilution therapy functions through lowering packed cell volume, reducing blood viscosity and increasing regional cerebral blood flow. According to literature reports, early normovolemic hemodilution and adhesive exudates with long half-life (such as synthesized protein Pentastarch which can carry oxygen or albumin etc.) are expected to show better effects. Selective blood component dilution is a new therapy based on hemodilution and ultraviolet blood irradiation therapy used worldwide. It changes or abandons plasma or abandons blood cells according to different kinds of blood rheology of ischemic brain damaged patients.

### 5. Vasodilative Medicine

Commonly used vasodilative medicines are cinnarizine, papaverine, calcium bicarbonate and calcium antagonist nimodipine, et al. However some people don't intent to use vasodilative medicines for they think application of them at the acute stage of ischemic cerebrovascular can cause reperfusion injury easily and aggravate brain tissue damages of penumbral area.

On the other hand, it has recently become a research hotspot of cerebral ischemic therapy to protect the ischemic brain tissue and neurons. Many neuroprotective agents are in clinical development. Main action pathways of neuroprotective agents for acute ischemic brain damage include: preventing influx of Ca²⁺, removing the free radicals, using excitatory amino acid receptor antagonist, neurotrophic factor, and γ-aminobutyric acid receptor agonist et al. More attention has been focused on the following pathways:

### 1. Ca²⁺ Channel Blocker

The earliest and most extensively studied Ca²⁺ channel blocker is dihydropyridine type medicines. Let's take nimodipine as an example. As voltage-sensitive calcium channel antagonist, nimodipine is lipophilic and easy to pass blood-brain barriers. Among randomized controlled study with 3719 patients, no significant effect was seen with 120 mg oral administration; the risk of bad result decreased 38% for patients receiving treatment within 12 hrs from onset. The effect of using nimodipine within 6 hrs is still in process of clinical III research in Netherlands. Small sample research of magnesium shows good patient tolerance; most patients treated showed neural functional improvement and less readmission within 6 months. Study from a group of 60 patients showed that after regular magnesium sulfate treatment, the mortality and morbidity rate for the treated group is 30% and the number is 40% for the placebo group and study of the exact effect of this medicine is still in process.

### 2. Free Radical Scavenger

Many free radicals are produced in acute cerebral ischemia and this will damage cell membrane and neurons. The free radicals also cause vasospasm and intravascular coagulation in the penumbral area, widen the infracted area and exacerbate the brain tissue damage. So it is crucial to scavenge the free radicals. Vitamin E, vitamin C, superoxide dismutase (SOD), hormone and mannitol etc are commonly used free radical scavenger and they can protect ischemic brain cells.

### 3. Glutamic Acid Release Inhibitor

Glutamic acid release inhibitors cure acute ischemic brain damage through inhibiting the synthesis and release of presynaptic glutamic acid. Animal experiments showed glutamic acid release inhibitors have obvious brain protection function but its clinical application is to be verified.

Researchers worldwide have always devoted to solve the problem of post-ischemia brain cell protection. For the pathological mechanism of neural tissue and cell damage induced by cerebral ischemia involves complex time-space cascade and reciprocal reactions, any interfering therapeutic test merely aiming at a single step or molecular mechanism is almost doomed to bring little effect. That is why most clinical tests are disappointing so far in spite of the promising therapeutic methods verified on animal models and constant effort for developing anti-cerebral ischemia chemicals. Maybe it is because of the neglect of the multistage, multi-center mechanism and multi-direction character of the cell damage process derived from cerebral ischemia.

On the whole, although human being has deeply understood the mechanism and pathological changes of cerebral ischemia, there is still a great need of relevant effective therapeutic means. So far only thrombolysis and recanalization are mainly used for cerebral ischemia. However these treatments only recovered the blood supply, there are still risk of hemorrhage and other limitations in application, and they are almost helpless for post-ischemic neuron damages especially reperfusion injury and delayed death. Although general efficacy is not ideal, the yearly expenditure on treating apoplexy reaches hundred billion RMB, and this doesn't include the huge cost for the long term brain damage recovery and neural functional maintain that may occur among the patient's whole life. Thus it is extraordinary important to find medicines interfering cerebral ischemia's development in multiple steps and mechanisms. These medicines will have a profound influence on therapy of cerebral ischemia, greatly reduce the spiritual and economic burden of the society, family and individuals, take a place in the great potential medicine market, and bring remarkable social and economic benefits.

Some pharmacologists predict promising effective anti-cerebral ischemia medicines would arise from chemicals that can bind multiple targets. Among the numerous research objects, neuroactive steroids are gaining increasing attention for their wide effect on nervous system.

Neuroactive steroids (NASs) is the general designation of all natural or synthesized neuroactive steroid hormones. NASs have the same typical action mode as general steroid hormones which regulate various receptors and proteins' gene expression in neuron and glial cells, which is called genomic effect. Genomic effect includes DNA transcription, translation and protein modification and it functions slowly. NASs can also quickly modulate activity of GABA receptor, Glu receptor, acetylcholine receptor and intracellular Sigma1 receptor, directly affect central activities (like emotion, neuronal plasticity and excitability, synaptic transmission, learning and memory, et al) in a non-genomic way. Recent research showed some NASs' have obvious modulation on neuronal damage, death, and variant central nervous system diseases. For example, kainic acid can cause hippocampus CA1 and CA3 neuron loss while allopregnanolone (AL) can relieve the loss. AL also showed such protection in irreversible neurotoxicity cell model cultured in vitro (low oxygen and Glu). Although dehydroepiandrosterone (DHEA) can decrease the neurotoxicity of Glu analogues and corticosteroid, its concentration in vivo decreases with age which is considered to correlate with neurodegenerative change accompanying aging. DHEA also showed such protection in reversible rabbit spinal cord ischemia model. Certain amount of DHEA can significantly increase in vitro rat embryo cortical center anoxia neuronal viability. Estrogen is known as the most powerful neuro-protective NAS. Consistent with Parkinson's Disease's (PD) gender differences, estrogen has fine protection on nigral dopaminergic neurotoxicity. Postmenopausal women stop secreting estrogen, which will cause increase of amyloid precursor protein in brain and sedimentary of Aβ, while Aβ's level decreases with administration of estrogen. This suggests the internal connection between estrogen and Alzheimer's Disease (AD). Estrogen can not only decrease neuronal death caused by ischemia, apoplexy and many other factors, but also postpone kainic acid induced epilepsy and reduce epilepsy caused hippocampal damage. Estrogen's obvious neuronal protection in several toxicity models suggests its various functions on receptor activation, antioxidation, antagonizing biological toxicity, regulating signal transduction pathways and gene expression, et al. Some researchs showed that estradiol and testosterone have similar neurotrophic function and they can promote regeneration after central nervous system damage. Like calcium channel antagonists, estradiol can decrease cerebral ischemic reperfusion injury, decrease and postpone animal's epilepsy attack. Estradiol and plant estrogen can decrease the central nerve system cholinergic neuron degeration in castrated rats, reduce the loss of basal forebrain cholinergic neuron and hippocampal cholinergic nerve fibers, and improve animal's cognitive ability.

### SUMMARY OF THE INVENTION

Marine steroid YC-1 (24-methylene-cholest-3β,5α,6β,19-tetrol) is an keto-sterol with uncommon side chain. Some documents show YC-1 can not only improve learning and memory ability, but also inhibit Glu induced cerebellar granule neuron excitotoxin death in a non-genomic dose dependent and non-NMDA receptor mediated way. YC-1's dose-dependent inhibition on low potassium induced cerebellar granule neuron apoptosis suggests YC-1 plays its role through inhibiting c-Jun's gene expression and reducing c-Jun's phosphorylation, and PI3K/AKT pathway may be involved in the process. YC-1 can also dose-dependently inhibit cerebellar granule neuron apoptosis induced by hypoxia. In whole animal experiment, both behavioral index and tissue's activity assay indicated YC-1 has obvious preventive and therapeutic effect on not only rat MCAO induced focal cerebral ischemia injury but also rabbit spinal cord ischemia injury. YC-1 and its analogue soft coral cholestene can indirectly show its antioxidant defense through repressing glutathione (GSH)'s exhaustion etc, and show its anti-inflammatory function through stabilizing membrane and decreasing inflammatory factor PEG2s' release. Thus YC-1 may be an effective anti-ischemic brain damage chemical aiming at multiple targets and through various mechanisms and a potential clinical medicine anti-ischemic brain damage. Excitotoxin, oxidative stress, inflammatory response, neuronal apoptosis and necrosis play an important role in development of neurodegenerative diseases like PD (Parkinson's Disease) and AD (Alzheimer's Disease). Thus, YC-1 is a greatly promising candidate of medicines treating neurodegenerative diseases.

The keto-sterols compounds marine steroid YC-1 extracted from Nephthea albida is protective to various neuron damages and showed no toxic reaction under effective protective dosage. The yield of YC-1 extracted from natural resources is limited and is not suitable for mass production. However synthesis can solve the problem. Starting with cheap hyodeoxycholic acid, YC-1 can be synthesized through 14 steps of organic reactions. Then the chemical is characterized with IR, ¹H NMR, ¹³C NMR, MS and EA.

This invention lays a firm foundation for optimizing and perfecting YC-1's structure and efficiency, finishing pre-clinical and clinical trials, and developing YC-1 to a new neuron protective medicine.

### BRIEF DESCRIPTION OF THE DRAWINGS

FIG. 1 represents the synthetic route of YC-1 (24-methylene-cholest-3β,5α,6β,19-tetrol).

FIG. 2 represents YC-1's ¹³C NMR spectra.

FIG. 3 represents YC-1's ¹H NMR spectra.

FIG. 4 represents YC-1's infrared (IR) spectra.

FIG. 5 represents YC-1's masspectrometry (MS).

FIG. 6 indicates YC-1 can reduce hemisphere infarct volume in permanent middle cerebral artery occlusion (MCAO).

FIG. 7 represents YC-1's protection on rats with permanent middle cerebral artery occlusion (MCAO).

FIG. 8 represents YC-1's effect on neurological function score of rabbit spinal cord ischemia model.

FIG. 9 represents YC-1's effect on behavior of rabbit spinal cord ischemia model.

FIG. 10 indicates YC-1 can increase the number of spinal cord anterior horn neurons after rabbit spinal cord ischemia.

FIG. 11 is a HE dye result indicating YC-1's effect on spinal cord anterior horn neurons after rabbit spinal cord ischemia.

FIG. 12 represents CGNs morphology revealed by phase contrast microscope after sequential treatment of YC-1 and H/R.

FIG. 13 is a FDA dye result of viable cells indicating YC-1 can inhibit H/R induced CGNs apoptosis.

FIG. 14 is the post-apoptosis Hoechst33258 nuclear morphology analysis indicating YC-1 can inhibit H/R induced CGNs apoptosis.

FIG. 15 represents CGNs morphology revealed by phase contrast microscope after sequential treatment of YC-1 and LK.

FIG. 16 is a FDA dye result of viable cells indicating YC-1 can inhibit LK induced CGNs apoptosis.

FIG. 17 is the post-apoptosis Hoechst33258 nuclear morphology analysis indicating YC-1 can inhibit LK induced CGNs apoptosis.

FIG. 18 is the agarose gel electrophoresis analysis indicating YC-1 can reduce DNA fragments produced from LK induced CGNs apoptosis.

FIG. 19 is a FDA dye result of viable cells indicating YC-1 can inhibit Glu induced CGNs apoptosis.

### DETAILED DESCRIPTION OF THE INVENTION

### 1. Synthesis of Marine Steroid YC-1

### 24-methylene-cholest-3β,5α,6β,19-tetrol

Starting with cheap hyodeoxycholic acid, YC-1 and some of it analogues were synthesized through 14 steps of organic reactions; then the intermediates and target chemicals were characterized by using spectrum technology.

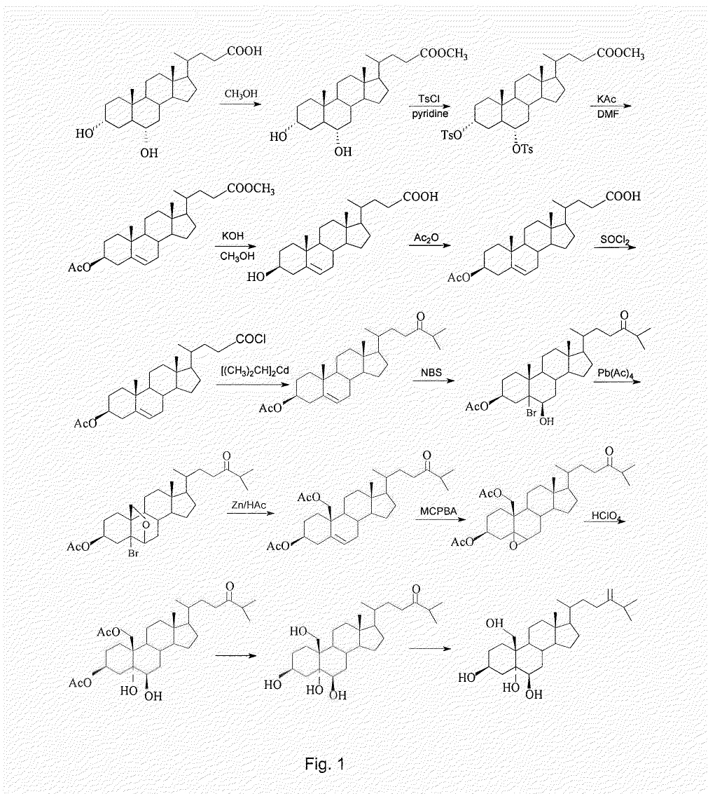

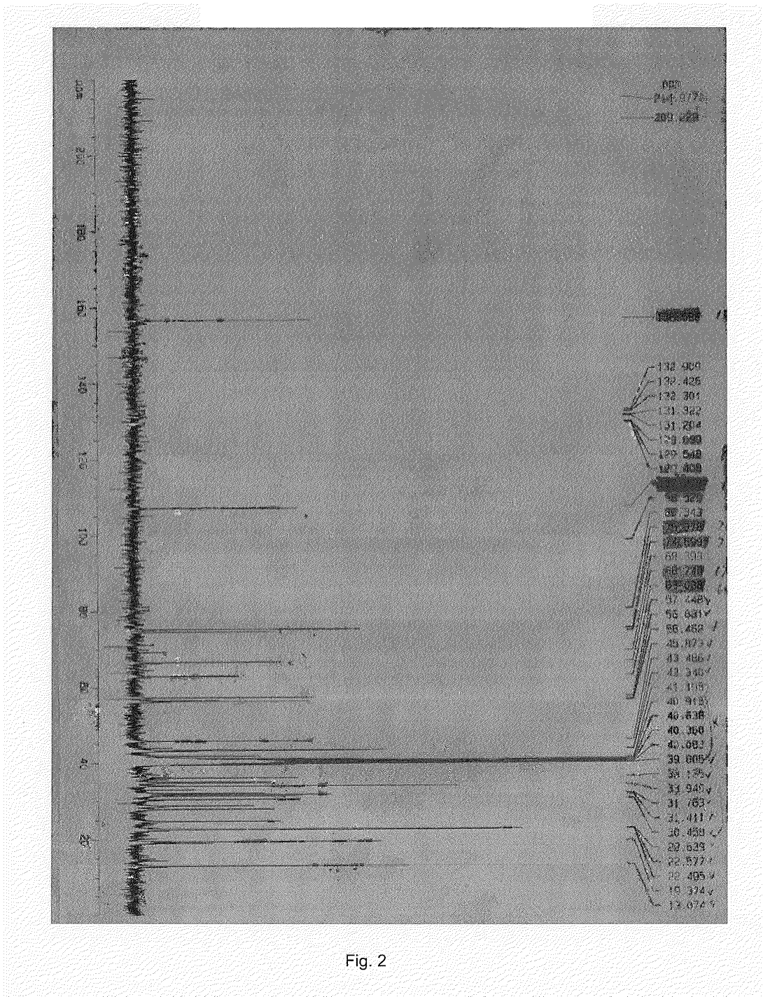

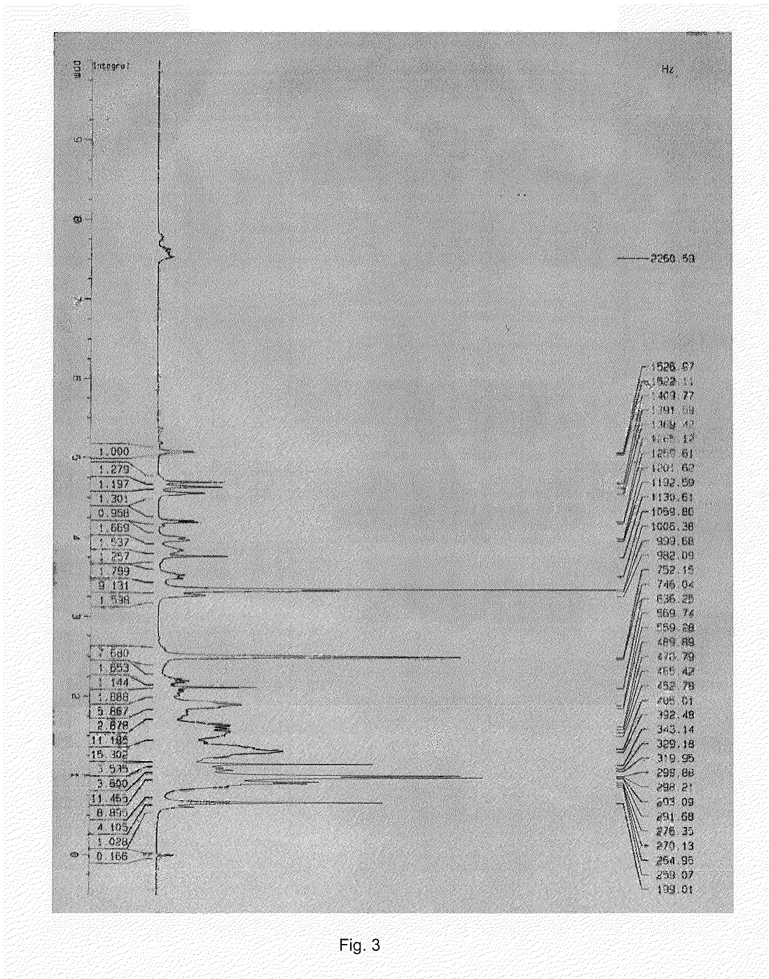

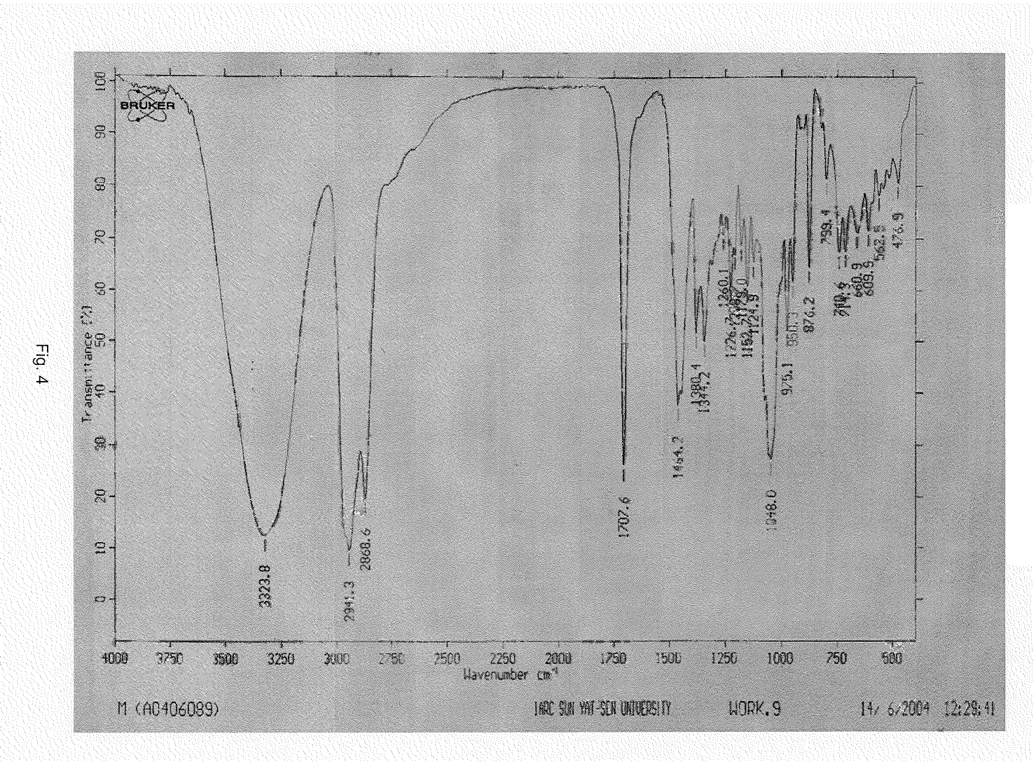

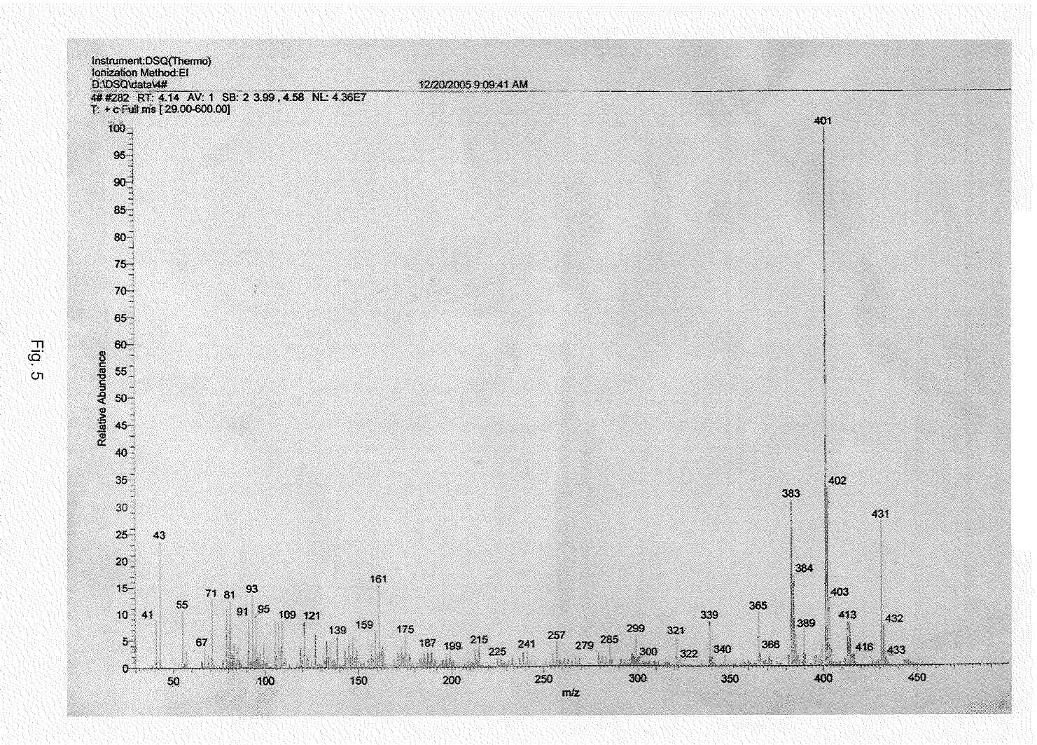

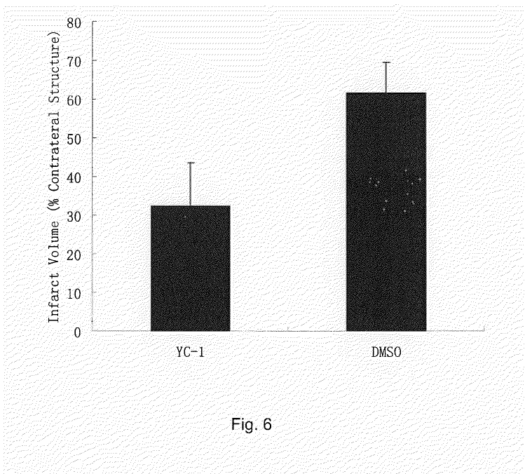

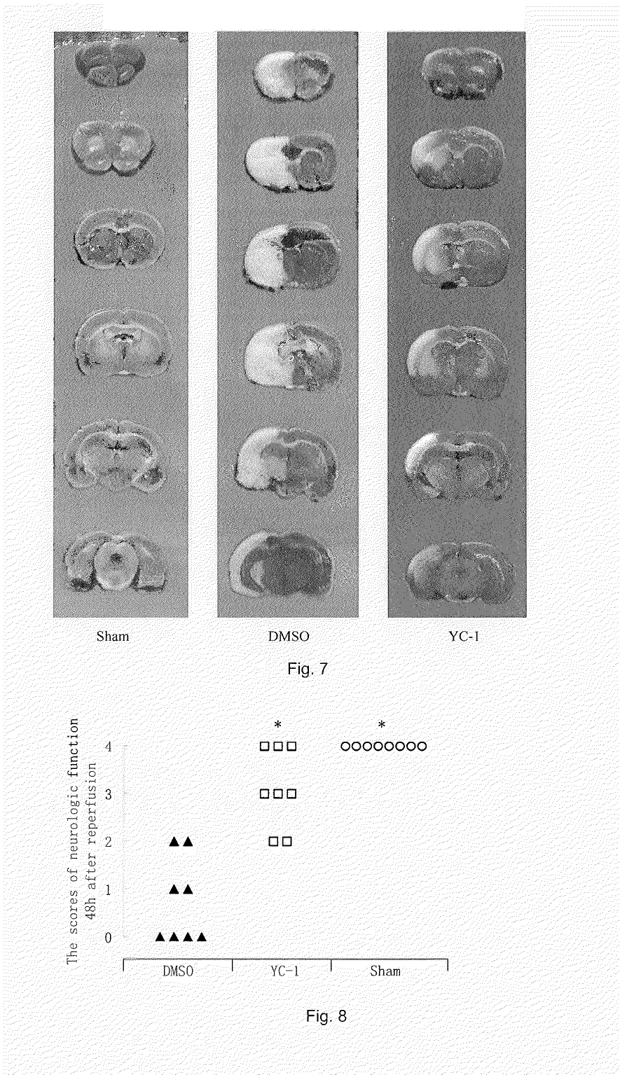

### 1.1 Synthetic Route of YC-1 (24-methylene-cholest-3β,5α,6β,19-tetrol), see FIG. 1

### 1.2 Optimization of YC-1's Synthetic Conditions

### 1.2.1 Synthesis of 3-acetoxy-cholest-5-Br-6-OH-24-ketone

It was synthesized through reaction of 3-acetoxy-cholest-5-ene-24-ketone and N-bromosuccinimide. The yield was low and there were multiple isomers in the product. The reaction was optimized by trying different conditions like solvents, temperature etc. Then different purification methods like recrystallization, solvent precipitation, chromatography etc. were tried.

### 1.2.2 Synthesis of 3-acetoxy-cholest-5-Br-6,19-epoxy-24-ketone

It was synthesized by remote oxidative ring-closure of 6-OH in 3-acetoxy-cholest-5-Br-6-OH-24-ketone. Lead tetraacetate and diacetyl iodobenzene were used for the oxidation under UV light and in ultrasonic wave respectively and also the reaction condition was optimized.

### 1.2.3 Synthesis of 3,19-diacetoxy-5,6-epoxy-24-ketone

It was done by oxidation of 5,6 double bond in 3,19-diacetoxy-5ene-24-ketone. Oxidation products got with different oxidant had different cis-trans ratio and yield. Totally there oxidants were used for the oxidation: m-chloroperoxybenzoic acid, hydrogen peroxide/formic acid system, and potassium permanganate/copper sulfate system. Then the reaction condition was optimized.

### 1.3 Characterization of Target Chemical YC-1 and the Intermediates

YC-1 and the intermediates were characterized with IR, ¹H NMR, ¹³C NMR, MS and EA. Among which YC-1's spectral data was:

Melting point: 223-225° C.

Elemental analysis: C28H4804

Measured value: C, 75.20; H, 10.36

Theoretical value: C, 74.95; H, 10.78

MS: [M-H₂O]⁺=432,

IR: 3416, 3028, 2936, 2868, 1642, 1464, 1377, 1057

¹H NMR (DMSO)

5.25 (1H, d, 6β-OH), 4.71 (1H, d, J=1.5 Hz, 28-CH), 4.65 (1H, d, J=1.5 Hz, 28-CH), 4.51 (1H, d, 19-OH), 4.16 (1H, d, 3-CH), 4.01 (1H, d, 19-CH), 3.84 (1H, m, 3-CH), 3.64 (1H, s, 5-OH), 3.23 (1H, m, 6-CH), 2.25 (1H, m, 25-CH), 1.13 (6H, d, 26-CH₃, 27-CH₃), 1.01 (3H, s, 19-CH₃), 0.87 (3H, d, 21-CH₃), 0.67 (3H, s, 18-CH₃).

¹³C NMR (DMSO)

156.6 (C), 107.3 (CH₂), 75.4 (C), 74.7 (CH), 66.7 (CH), 63.0 (CH₂), 57.4 (CH), 56.7 (CH), 45.9 (CH), 43.5 (C), 43.3 (CH), 42.8 (C), 41.3 (CH₂), 40.9 (CH), 37.4 (CH₂), 36.1 (CH), 33.9 (CH₂), 31.8 (CH₂), 31.4 (CH₂), 30.5 (CH₂), 28.7 (CH₂), 27.9 (CH₂), 24.70 (CH₂), 22.64 (CH₃), 22.57 (CH₃), 19.4 (CH₃), 13.1 (CH₃).

### 1.4 Design and Synthesis of YC-1's Analogues

(1) 3-hydroxycholest series analogues

(2) 3,6-dihydroxycholest series analogues

(3) 3,5,6-trihydroxycholest series analogues

(4) 3,19-dihydroxycholest series analogues

(5) 3,5,6,19-tetrahydroxycholest series analogues

(6) 3-hydroxycholest dimer

(7) 3,5,6,19-tetrahydroxycholest dimmer

(8) The side chain at position 17 of chemicals in (1)-(7) can be: 24-carbonyl, 24-methylene, 24-hydroxy or 24-hydrazone etc. Then they were characterized by using techniques like IR, NMR, MS and EA etc.

### 1.5 Study of Steroid Structure Modification

YC-1 has poor water solubility. To improve its solubility, the 3-hydroxy group in 3-hydroxy-cholest-5-ene-24-ketone went through hydrophilic modification.

### 1.5.1 Synthesis of cholest-5-ene-24-ketone-3-hydroxy-sulfonic ester

### 1.5.2 Synthetic study of PEG modified 3-hydroxy-cholest-5-ene-24-ketone

### 2. YC-1 and its analogues' effect on MCAO induced rat cerebral ischemia model

### Method: Preparation of Rat Middle Cerebral Artery Occlusion (MCAO) Model

25 healthy male SD rats were divided into 3 groups randomly:
YC-1 group (n=11): 10 min, 6 h, 24 h and 7 d after occluding the middle cerebral artery, 12 mg/Kg YC-1 was continuously injected intraperitoneally.
DMSO group (n=11): same volume of DMSO (1 ml/Kg) was injected in the same way.
Sham group (n=3): just very short intraluminal thread was inserted and didn't occlude the MCA.

The following method was a modification of Zea Longa's method (Longa E Z, Weinstein P R, Carlson S, Cummins R. Reversible middle cerebral artery occlusion without craniectomy in rats. Stroke, 1989, 20: 84-91).

(1) The rats drinked freely but fasted one night before the operation.

(2) 10 min before anesthesia, atropine sulfate (0.5 mg/Kg) was intraperitoneally (ip) injected.

(3) Narcotized the rat with ip injected 10% chloral hydrate, 3.5 ml anesthetic per Kg rat; The areflexia of hindlimb retraction and corneal were used as anesthesia index.

(4) The rat was supinely fixed after anesthesia, the cervical skin and subcutaneous tissue were cut, left cervical skin was separated from digastric, sternocleidomastoid and omohyoid, the common carotid artery (CCA) bifurcation was exposed, including the internal and external carotid artery (ICA, ECA) extended from CCA bifurcation in the triangle formed by the three muscles.

(5) The CCA and ECA were ligated at the beginning of ascending pharyngeal artery as follows: a thread was prepared at the proximal end of ICA, CCA was incised at the bifurcation, a 5 cm nylon intraluminal thread was inserted to ICA's proximal end, stopped when inserted about 17-18 mm and felt resistance. At this moment the nylon thread entered ICA and anterior cerebral artery, all blood sources for MCA were blocked, and it was timed immediately when blocking the blood flow.

(6) The prepared thread was fastened at proximal ICA, the redundant thread was trimmed, disinfected with iodine tincture, the skin was sutured and the rat was returned back to cage.

(7) Rectum temperature was measured by using Meikangti electronic temperature measure instrument the whole process and it was strictly controlled at 36.5-37.5°.

(8) Except using shorter intraluminal thread (10 mm) which didn't reach MCA, all other steps for the Sham group were the same as above.

Result: By visual inspection, the brain tissue in the Sham group had normal color and morphology with no pallor and swelling; those in the DMSO group showed obvious pallor and swelling and no obvious pallor and swelling in the YC-1 group. TTC dye showed that YC-1 group had smaller infarct volume than DMSO group (the infarct size was 32±11% and 62±8% respectively) while no ischemia was observed in the Sham group.

### 3. YC-1's Effect on Abdominal Aorta Clipping Induced Rabbit Spinal Cord Ischemia Model

Method: Preparation of spinal cord ischemia model.

24 male New Zealand white rabbits were divided evenly into 3 groups (n=8):

YC-1 group: 30 min before spinal cord ischemia, 8 mg/Kg YC-1 was intravenous injected through the rabbit's ear.
DMSO group: same volume of DMSO (1 ml/Kg) was injected in the same way.

Sham group: the abdominal aorta was exposed but wasn't clipped.

Referring to Johnson etc, the model was prepared as follows:

(1) The rabbits drinked freely but fasted one night before the operation.

(2) The rabbit was anesthetized by injecting 3% sodium pentobarbital (30 mg/Kg) by ear intravenous. On the other side of the ear a vein-detaining needle (24 G) was set for medicine and other fluid infusion.

(3) An artery-detaining needle (24 G) was set in the right ear artery for monitoring the proximal arterial pressure and blood sampling during the operation.

(4) An artery-detaining needle (24 G) was set in a femoral artery for monitoring the distal arterial pressure during the operation.

(5) The rabbit was supinely fixed and incised at the median abdomen; the abdominal aorta was exposed.

(6) Injected heparin (150 U/Kg) through the vein-detaining needle, then the abdominal aorta was clipped at the 0.5-1.0 cm spot below origin of the left renal artery to cause spinal cord ischemia.

(7) Once the abdominal aorta was blocked, the distal arterial pressure decreased immediately and the femoral arterial pulsation disappeared.

(8) The clip was removed 20 min after ischemia and the abdomen was sutured.

(9) Then intramuscularly injected 40,000 U gentamicin, the rabbit was returned back to cage and observed for 48 h.

(10) The proximal arterial pressure, distal arterial pressure and heart rate (Spacelab, US) were continuously monitored during the operation. Rectum temperature was maintained at 38.5±0.5° by using heating pad and roasting lamp.

(11) Blood gas (AVL-2, Switzerland) and glucose (One Touch II, USA) were monitored by ear artery blood sampling 10 min before ischemia, 10 min after ischemia and 10 min after reperfusion.

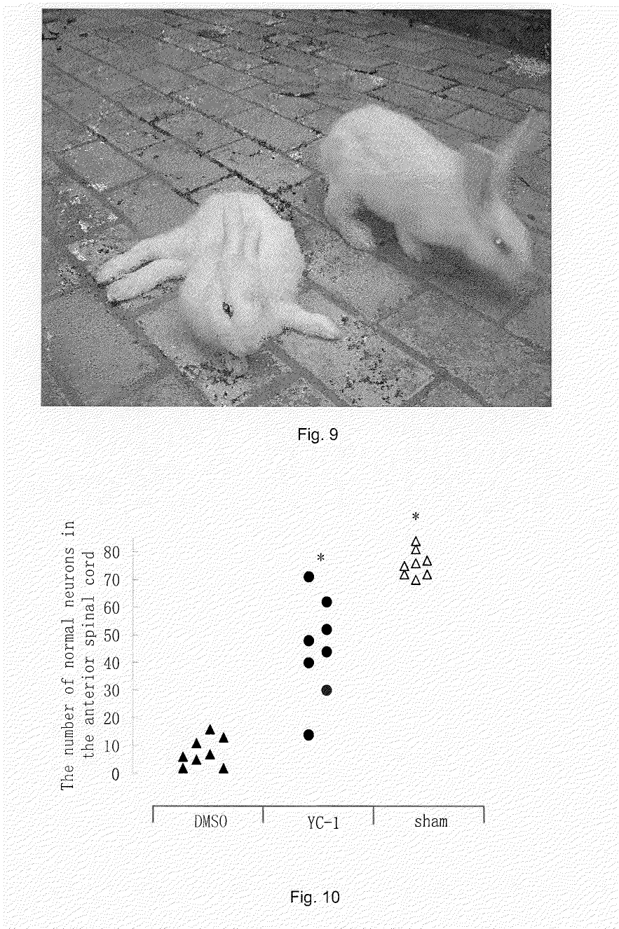

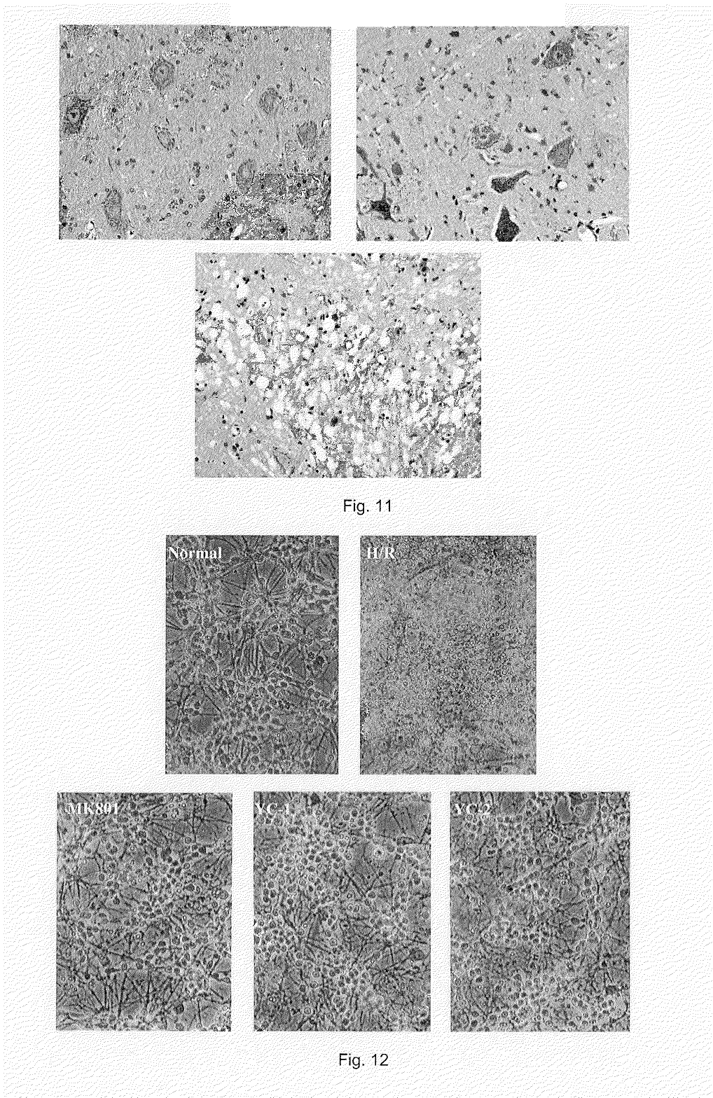

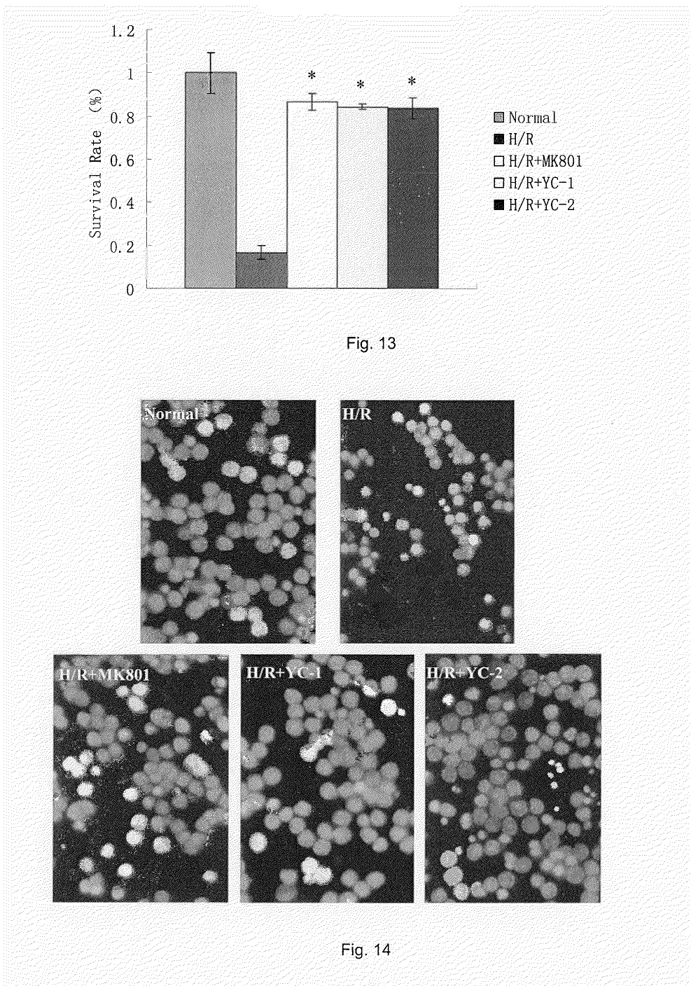

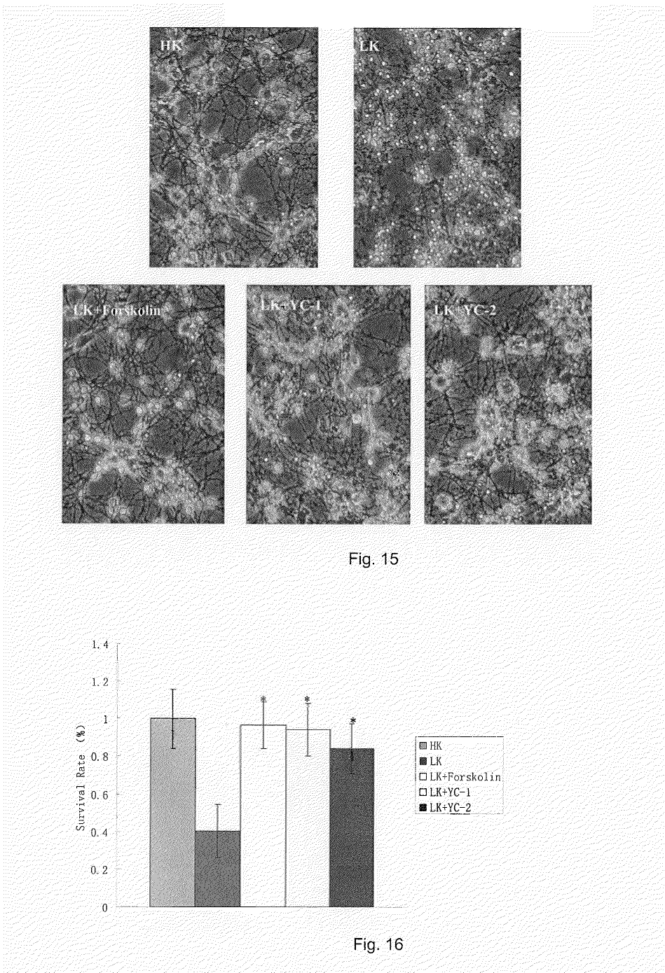

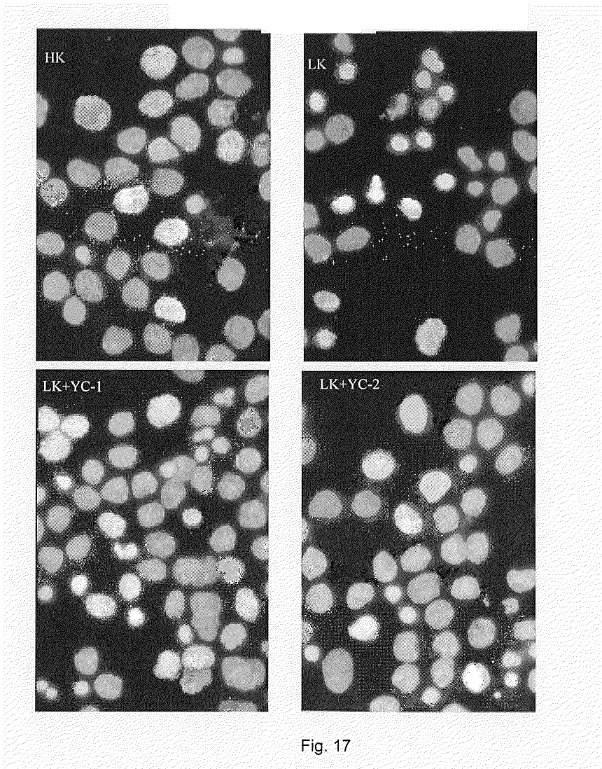

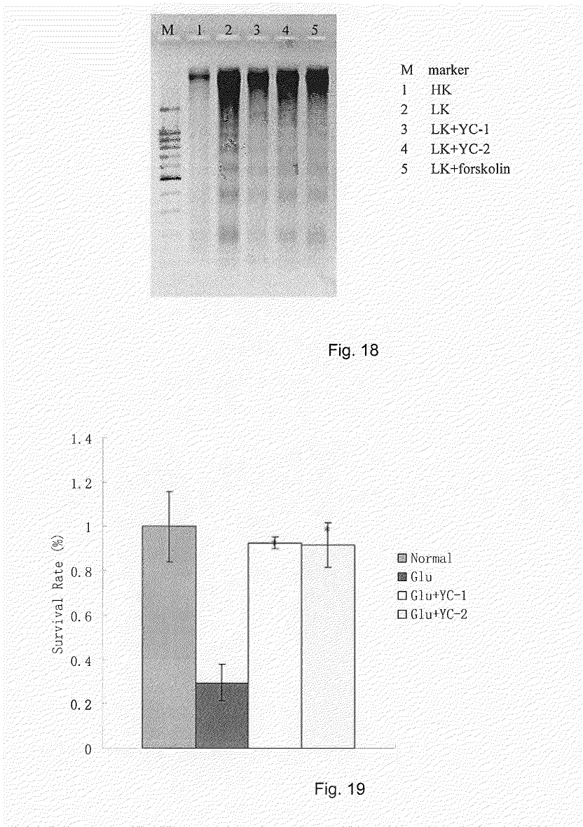

Result: See FIG. 8 for neurological function score of animals from each group. Hindlimb nerve in the sham group functioned normally (4 points) during the whole observation period. None rabbit in DMSO group could stand. Six rabbits in the CPA (YC-1) group could stand and the other two rabbits' hindlimbs also showed obvious movement. At each observation time point, neurological function score in YC-1 and sham groups were obviously higher than that in DMSO group (p<0.05). Histopathological examination showed that the lumbar spinal cord in DMSO group was severely injured manifesting as reduction of motor neurons, disappearance of niss1 bodies and nucleus and vacuolar degeneration, while lumbar spinal cord injury in YC-1 group was obviously relieved, see FIG. 9. Comparing with DMSO group, the number of normal spinal cord anterior horn neurons in CPA (YC-1) and sham groups increased evidently (p<0.01), as shown in FIG. 10.

### 4. YC-1 and its Analogues' Effect on Hypoxia Induced In Vitro Cultured Cerebellar Granule Neurons Death

### Method:

1) Culture of Rat Cerebellar Granule Neurons (CGNs)

According to Yan, etc, the neurons were prepared as follows:

(1) A clean 7-8 d SD rat weighing about 15-20 g was taken and the cerebella was separated under sterile condition.

(2) The meninges and vessels were gotten rid of in Kreb's anatomy solution (Ca²⁺ and Mg²⁺ free).

(3) The cerebella was sheared into pieces of 1 mm³ with ophthalmic scissors.

(4) Digested in 0.25 g/L trypsin solution for 15 min at 37.

(5) The digestion was stopped with solution containing 0.5 g/L trypsin inhibitor and 0.05 g/L Dnase I and pipetted up and down to get single cell suspension.

(6) The single cell suspension was centrifuged at 200 g for 5 min, the precipitation was rinsed once with rinse solution.

(7) Span down again at 200 g for 5 min, the supernatant was discarded.

(8) The cell pellet was diluted to cell density of 1.5-1.8×10⁶ cells/ml with BME medium containing 10% (v/v) FBA and 25 mM KCl.

(9) They were inoculated into cell culture dishes pre-coated with polylysine and incubated at 370 incubator with 5% CO₂.

(10) Twenty-four hours after inoculation, 10 μmol/L Ara-C was added to inhibit non-neuron cells' growth and proliferation and to make the purity of CGNs above 95%.

(11) Glucose was added to 5 mM on day 7 incubation to supplement energy needed by cell metabolism.

(12) The experiment on day 8.

2). Establishment of CGNs Hypoxia/Reoxygenation (H/R) Injury Model

Referred to Seko (Seko Y, To be K, Ueki K, et al. Hypoxia and hypoxia/reoxygenation activate Raf-1, mitogen-activated protein kinase kinase, mitogen-activated protein kinases, and S6 kinase in cultured rat cardiac myocytes. Circ Res. 1996 January; 78(1):82-90) etc for the establishment method:

(1) On day 8, the treated CGNs were put in airtight box with oxygen consumption reagent and indicator and incubated at 37° for 3 h.

(2) The cells were transferred to 370 incubator with 5% CO₂ for reoxygenation treatment.

(3). Cell morphological observation and neuron viability measurement

The following were done referring to Yan (Yan G M, Irwin R P, Lin S Z, et al. Diphenylhydantoin induces apoptotic cell death of cultured rat cerebellar granule neurons. J Pharmacol Exp Ther, 1995, 274(2): 983-8) et al.:

(1) CGNs were cultured in 35 mm dishes, on day 8, the medium was removed after various factors treatment, the cells were rinsed twice with 4 PBS containing Ca²⁺ and Mg²⁺.

(2) The cells were fixed with 40 g/L paraformaldehyde (40, dissolved in PBS) for 10 min.

(3) The fixation solution was removed, the cells were rinsed twice with distilled water, air dried at 4°.

(4) The cell morphological change was observed and randomly photographed under inverted fluorescence microscope (Olympus).

### Result: 1) Result Got Under Inverted Fluorescence Microscope:

(1) Before hypoxia, CGNs cell bodies were plump and lucent; Cell processes and the network formed by them were tight, clear and intact.

(2) After 3 h hypoxia and 24 h reoxygenation (H/R) treatment, the CGNs cell body dwindled; the structure became unclear; the processes and network showed disorder and fracture.

(3) The cells were first treated with 10 μM MK801 or 12 μM YC-1 or 12 μM YC-2 for 3 h, then the H/R treatment and observation were done. Most CGNs cell body kept intact, plump and lucent; the processes and network were tight, clear and intact.

2) FDA Dye Analysis:

(1) After H/R treatment, neuronal viability decreased obviously (16.56±3.21%).

(2) After treated by 10 μM MK801 or 12 μM YC-1 or 12 μM YC-2, neuronal viability reached 86.73±3.21%, 84.29±1.26% and 83.54±4.78% respectively.

3) Hoechst33258 Dye Analysis:

(1) Normal CGNs had intact nucleus and the chromatin distributed evenly.

(2) After H/R treatment, CGNs nucleus and chromatin pyknosis together with apoptotic bodies were observed.

(3) The cells were first treated with 12 μM YC-1 YC-2, then the H/R treatment and observation were done. Most CGNs showed normal nuclear morphology and even chromatin distribution.

### 5. YC-1 and its Analogues' Effect on Repolarization Induced In Vitro Cultured Cerebellar Granule Neurons Death

### Method:

The methods for rat CGNs cell culture, cellular morphologic observation and neuronal viability measurement were the same as above. Then check the in vitro cultured cerebellar granule neurons death through agarose gel electrophoresis according to reference:

(1) CGNs in 35 mm² dishes were inoculated.

(2) On day 8, 24 h after adding various medicines, the medium was removed, the cells were rinsed twice with PBS.

(3) The collected cells were spun down with a low temperature high speed centrifuge (Biofuge22R, Heraeus, Germany) at 5000 rpm, 4° for 5 min.

(4) the cell pellet was resuspended with 600 μl buffer containing 10 mmol/L Tris-HCl, 10 mmol/L EDTA and 2 g/L Triton X-100 (pH 7.5) and incubated for 15 min.

(5) Span down at 12000 rpm, 40 for 10 min.

(6) The supernatant was extracted once with equal volume of phenol and spun down.

(7) The supernatant was extracted once with equal volume of phenol-chloroform (1:1), spun down.

(8) The supernatant was taken, 300 mmol/L Sodium Acetate and equal volume of isopropanol were added and precipitated for overnight.

(9) The pellet was spun down and rinsed 2-3 times with 70% ethanol.

(10) After drying, some TE (Tris 10 mmol L⁻¹, EDTA 1.0 mmol L⁻¹, pH 7.4, and 0.6 g·L⁻¹ RNase A) were added and incubated at 37° for 30 min.

(11) The samples were analyzed with 20 g/L agarose gel electrophoresis for 1 h.

(12) Dyed with ethidium bromide and photographed under UV light.

### Result:

(1) Rat CGNs in vitro cultured in serum free BME medium containing 25 mM KCl for 8 days showed intact cell bodies and processes.

(2) Above cells were transferred to serum free medium with 5 mM KCl and incubated for another 24 h, the cell bodies dwindled and the nucleus fractured; FDA fluorescence staining indicated only 40.2±14.04% CGNs survived.

(3) If 12 μM YC-1 or YC-2 were added 3 h before and the same time changing to low potassium medium, then the cells were cultured in the low potassium medium for another 24 h, most CGNs kept intact cell bodies and processes; FDA staining indicated 94.3±14.02% and 84.2±13.34% CGNs survived respectively.

(4) These results showed marine steroid YC-1 and YC-2 obviously improved rat CGNs' viability in low potassium medium.

### 6. YC-1 and its Analogues' Effect on Glutamic Acid (Glu) Induced In Vitro Cultured Cerebellar Granule Neurons Death

### Methods:

(1) Isolatation and primary culture of rat CGNs.

(2) Morphology observation of neuron cell body and processes under phase contrast microscopy.

(3) Analysis of neuronal metabolic activity and viability using fluorescein diacetate (FDA) staining.

### Result:

(1) rat CGNs in vitro cultured for 8 days were taken, 150 μM Glu was added, then incubated for 24 h and observed under phase contrast microscopy. The neuronal cell bodies dwindled obviously, the nucleus showed pyknosis and the processes fractured or disappeared.

(2) If 12 μM YC-1 or YC-2 were added 1 h before adding Glu, then observed 24 h later. The result showed that both YC-1 and YC-2 could obviously inhibited Glu induced CGNs apoptosis.

(3) CGNs' Viability analysis using FDA staining showed that 12 μM YC-1 and YC-2 clearly improved CGNs' viability (respectively 92.6±2.59% and 91.6±10.11%) comparing to the Glu control group (29.5±8.23%).

1-2. (canceled)

3. A method for treating a human that has a disease state that is alleviated by treatment with a neuron protective agent, the method comprising:
administering a therapeutically effective amount of a marine steroid, or a pharmaceutically acceptable salt thereof, to a human in need thereof.

4. The method of claim 3, wherein the marine steroid is 24-methylene-cholest-3β,5α,6β,19-tetrol.

5. The method of claim 4, wherein the marine steroid is extracted from an animal.

6. The method of claim 4, wherein the marine steroid is extracted from Nephthea albida.

7. The method of claim 4, wherein the marine steroid is synthetic.

8. The method of claim 3, wherein the disease state is cerebrovascular disease or neurodegenerative disease.

9. The method of claim 8, wherein the cerebrovascular disease is cerebral ischemia.

10. The method of claim 8, wherein the neurodegenerative disease is Parkinson's disease or Alzheimer's disease.

11. The method of claim 3, wherein the marine steroid alleviates the disease state in a genomic way.

12. The method of claim 3, wherein the marine steroid alleviates the disease state in a non-genomic way.

13. A pharmaceutical composition, comprising:
a marine steroid or a pharmaceutically acceptable salt thereof; and
a pharmaceutical acceptable carrier.

14. The pharmaceutical composition of claim 13, wherein the marine steroid is 24-methylene-cholest-3β,5α,6β,19-tetrol.

15. The pharmaceutical composition of claim 13, wherein the marine steroid is extracted from an animal.

16. The pharmaceutical composition of claim 15, wherein the marine steroid is extracted from Nephthea albida.

17. The pharmaceutical composition of claim 14, wherein the marine steroid is synthetic.

18. A composition, comprising:
a marine steroid or a pharmaceutically acceptable salt thereof.

19. The composition of claim 18, wherein the marine steroid is 24-methylene-cholest-3β,5α,6β,19-tetrol.

20. The composition of claim 19, wherein the marine steroid is extracted from an animal.

21. The composition of claim 20, wherein the marine steroid is extracted from Nephthea albida.

22. The composition of claim 19, wherein the marine steroid is synthetic.
